(19) United States
(12) Patent Application Publication
(10) Pub. No.: US 2005/0260195 A1
(43) Pub. Date: Nov. 24, 2005

(54) Treatment of hepatitis B virus infection with human monoclonal antibodies

(75) Inventors: Shlomo Dagan, Nes-Ziona (IL); Rachel Eren, Netaim (IL)

Correspondence Address: HELLER EHRMAN WHITE & MCAULIFFE LLP, 1717 RHODE ISLAND AVE, NW, WASHINGTON, DC, 20036-3001, US

(73) Assignee: XTL BIOPHARMACEUTICALS LTD., Rehovot (IL)

(21) Appl. No.: 11/056,186

(22) Filed: Feb. 14, 2005

Related U.S. Application Data
(63) Continuation-in-part of application No. 10/491,660, filed on Jul. 22, 2004, now abandoned.

(51) Int. Cl. A61K 39/42; C12Q 1/70

(52) U.S. Cl. 424/141.1; 424/161.1

(57) ABSTRACT

Disclosed is a pharmaceutical composition for the treatment or prevention of hepatitis B virus infection, comprising a 1:3 mixture of two fully human anti HBsAg monoclonal antibodies 19.79.5 and 17.1.41. Also disclosed are preferred modes of administration. The pharmaceutical composition can be given as a monotherapy or in combination with other anti viral agents.

### FIELD OF THE INVENTION

The present invention concerns a pharmaceutical composition for the treatment or prevention of hepatitis B infection comprising a mixture of two human monoclonal antibodies.

### BACKGROUND OF THE INVENTION

Despite introduction of universal vaccination against hepatitis B in over 100 countries, persistent HBV infection is still a serious problem worldwide, causing an estimated annual death rate of one million (Kane, Lancet 1996; 348-696). It may take several decades until the effect of vaccination will be translated into reduced transmission and morbidity. Meanwhile, patients with persistent HBV infection require better anti-viral therapeutic modalities than are currently available. In the U.S., approximately 300,000 new cases of acute HBV infection occur annually, 10% of whom will become HBV carriers, and 50% of those will develop chronic liver disease with an increased risk for developing hepatocellular carcinoma (HCC) (El-Serag and Mason, N Eng J Med 1999; 340 745-750). Hepatitis B vaccines are effective in preventing primary infection but have not shown a significant effect in infected patients.

Two therapies are currently approved for treatment of chronic HBV infection: interferon alfa-2b (IFNα) (Wong et al., Ann Intern Med 1993; 119, 312-323) and lamivudine (Dienstag et al., N Eng J Med 1999; 341, 1256-1263). Both therapies provide only a partial solution to the disease due to a relatively low response rate, severe side effects of IFNα, and development of lamivudine resistant strains (Liaw et al., Hepatology 1999; 30, 567-572).

Passive immunotherapy utilizing preparations of human hyperimmune immunoglobulin from HBV-immune patients is commonly used as prophylaxis against liver re-infection after liver transplantation. It is given intramuscularly to neonates to prevent vertical transmission of HBV from infected mothers. It is not used for treatment of chronic patients.

Overall, the use of plasma-derived polyclonal antibodies is limited because these preparations have variable activity, limited availability and there are potential hazards for the transmission of infectious agents.

In contrast, monoclonal antibodies (mAbs) can be consistently produced and do not carry the infectious risks associated with plasma-derived products. Previous studies using a single human mAb for treating HBV-infected patients undergoing liver transplantation resulted in emergence of escape mutants (McMahon et al., 1992 Hepatology 15 (5) 757-766). The same antibody was administered for a two-week period to chronic hepatitis B patients pre-treated with lamivudine and was shown to form complexes with HBsAg and to reduce its level in patients. Three months after therapy HBsAg levels had returned to pre-treatment levels (Heijtink et al., 2001 J. Med. Virol. 64 427-434).

In another study, two fully human monoclonal antibodies were developed directed against different epitopes of hepatitis B surface antigen (HBsAg) (PCT/IL97/00184 and PCT/IL97/00183). A single administration of a mixture of these antibodies into HBV chronic carrier chimpanzees resulted in immediate reduction in HBsAg levels followed by a recurrence to initial levels within a few days (Eren et al., 2000 Hepatology 32, 588-596).

### SUMMARY OF THE INVENTION

In accordance with the present invention a pharmaceutical composition is provided comprising a combination of two, fully human, high-affinity monoclonal antibodies directed against different epitopes of hepatitis B virus surface antigen (HBsAg). In accordance with one embodiment of the present invention, a pharmaceutical composition is provided (designated HBV-Ab^{XTL}) comprising as an active ingredient a mixture of the human monoclonal antibody 19.79.5 as well as fragments thereof retaining the antigen binding characteristics of the antibodies, and the human monoclonal antibody 17.1.41 as well as fragments thereof retaining the antigen binding characteristics of the antibodies together with a pharmaceutically acceptable carrier. Antibody 19.79.5 is secreted by the hybridoma cell line deposited in the European Collection of Cell Cultures (ECACC) under Accession No. 96052168, and antibody 17.1.41 secreted by the hybridoma cell line deposited in the ECACC under Accession No. 96052169. Antibodies 19.79.5 and 17.1.41 are further characterized by their sequence disclosed in PCT/IL97/00184 and PCT/IL97/00183. Fragments retaining the antigen binding characteristics of the antibodies may be, for example, Fab or F(ab)₂ fragments obtained by digestion of the whole antibody with various enzymes as known and described extensively in the art. The antigenic characteristics of an antibody are determined by testing the binding of an antibody to a certain antigenic determinant using standard assays such as RIA, ELISA, or FACS analysis. Further aspects of the present invention are various prophylactic and therapeutic uses of the antibody mixture. In accordance with this aspect of the invention, the pharmaceutical composition comprising the antibody mixture may be used for the treatment of chronic Hepatitis B patients by administering to such a patient a therapeutically effective amount of the mixture of antibodies or fragments thereof capable of binding to the HBVsAg being an amount effective in alleviating the symptoms of the HBV infection or reducing the number of circulating viral particles in an individual. Means to assess alleviation of symptoms of HBV infection may include as a non limiting example measurement of liver functions by determining levels of the enzyme alanine aminotransferase (ALT) or by measuring sero conversion namely disappearance of the HBeAg or by examining liver biopsies and determining the level of tissue fibrosis by methods well known in the art. The number of circulating viral particles can be determined for example by measuring HBV DNA levels using PCR or by detecting HBsAg levels in the blood.

In one embodiment of the present invention the pharmaceutical composition is given in a dose ranging from 0.26 mg to 80 mg. Preferably 10 mg or 40 mg.

In a preferred embodiment of the present invention the pharmaceutical composition comprises an approximate ratio of 1:3 between antibodies 19.79.5 and 17.1.41 respectively.

In addition to the antibody mixture the pharmaceutical composition of the invention may optionally also comprise a carrier selected from any of the carriers known in the art. One example of such a carrier is a liposome. The pharmaceutical composition of the invention may also comprise various diluents and adjuvants known per se. The composition of the invention may be administered by a variety of administration modes including intra venous, intra muscular and subcutaneous administration. The pharmaceutical composition of the invention may be administered in combination with other anti-viral agents. Such agents may include, as a non-limiting example: interferons, anti hepatitis B monoclonal antibodies, anti hepatitis B polyclonal antibodies, nucleoside analogues, inhibitors of DNA polymerase and therapeutic vaccines. In case of such a combination therapy the antibodies may be given simultaneously with the anti viral agent or sequentially either before or after treatment with the anti viral agent.

The pharmaceutical composition of the invention may also be used, for example as a prophylactic treatment of neonates born to HBV infected mothers or of healthcare workers exposed to the virus or of liver transplant recipients to eliminate possible recurrent HBV infection of the transplanted liver.

### BRIEF DESCRIPTION OF THE DRAWINGS

FIG. 1: HBsAg and HBV-DNA serum levels of two patients infused with a single dose the HBV-Ab^{XTL} mixture. The HBV-Ab^{XTL} mixture was administered at time point 0. The time range is not to scale. A: patient no. 303, dose 0.26 mg, Ab:Ag molar ratio=1:14; B: patient no. 310, dose 39 mg, Ab:Ag molar ratio=1:2.

HBV-DNA [symbol] HBsAg [symbol] .

FIG. 2: HBsAg and HBV-DNA serum levels in four patients administered with multiple infusions of the HBV-Ab^{XTL} mixture. The HBV-Ab^{XTL} mixture was administered at time points (days) 0, 8, 15 and 22; arrows indicate administration time. A: patient no. 303, dose 4×10 mg; B: patient no.308, dose 4×20 mg; C: patient no. 105, dose 4×40 mg; D: patient no. 301, dose 4×80 mg.

HBV-DNA [symbol] HBsAg [symbol] .

FIG. 3: HBsAg and anti-HBsAg antibody serum levels in four patients administered with multiple infusions of the HBV-Ab^{XTL} mixture. The HBV-Ab^{XTL} mixture was administered at time points (days) 0, 8, 15 and 22; arrows indicate administration time. A: patient no.303, dose 4×10 mg; B: patient no. 308, dose 4×20 mg; C: patient no.105, dose 4×40 mg; D: patient no.301, dose 4×80 mg.

HBsAg [symbol] anti-HBsAgAb [symbol] .

Reference will now be made to the following Examples that are provided by way of illustration and are not intended to be limiting to the present invention.

### EXAMPLES

### Materials and Methods

Virological and Immunological Assays

Serum HBsAg levels. HBsAg levels were determined by a modified automated immunoassay (IMX system, Abbott GmbH Diagnostika) using a purified HBsAg preparation (Bio-Hep-B, Biotechnology General, Ness-Ziona, Israel) as standard.

Serum anti-HBs levels. Anti-HBs levels were determined by AUSAB RIA and compared to a WHO reference for anti-HBs. A reference serum for anti-HBs was obtained from CLB, Red Cross Blood Transfusion Service, the Netherlands.

Serum HBV-DNA levels. HBV-DNA levels in patients' serum were analyzed by HBV-DNA PCR using the Amplicor HBV Monitor™ Test (Hoffman-La Roche Inc., Roche Diagnostics, Branchburg, N.J., USA) according to the manufacturers' instructions.

Preparation of HBV-Ab^{XTL}

Each dose of HBV-Ab^{XTL} is prepared by diluting the two antibodies 19.79.5 and 17.1.41 in 250 ml normal saline solution in an approximate ratio of 1:3 between the antibodies respectively (i.e. for each mg of antibody 19.79.5 approximately 3 mg of antibody 17.1.41 are added).

### Example 1

HBV-Ab^{XTL} was first tested in a dose escalation (single-dose) phase IA study in patients with otherwise untreated chronic Hepatitis B infection (Galun et al., 2000 Hepatology 32 (4 Pt.2): p221A). A total of 15 patients were enrolled in the study and each received a single dose of HBV-Ab^{XTL}. The doses ranging between 0.26 to 40 mg. The dosing levels, were based on the molar ratio of antibody to antigen (Ab:Ag) (Table 1). HBV-Ab^{XTL} was administered as intravenous infusions over 2-8 hours.

TABLE 1
Pre-treatment clinical characterization of patients in phase 1A

| | | Dose | Ab:Ag | ALT | HBsAg | HBV-DNA |
|---|---|---|---|---|---|---|
| Patient | Cohort | (mg) | Molar ratio | (U/L) | (μg/ml) | (copies/ml) |
| 301 | I | 0.26 | 1:700 | 106 | 5.5 | 1.7 × 10⁷ |
| 302 | I | 0.26 | 1:600 | 10 | 3.7 | 3.5 × 10⁷ |
| 304 | I | 0.26 | 1:800 | 59 | 6.2 | 7.1 × 10⁶ |
| 303 | II | 0.26 | 1:14 | 15 | 0.1 | 2.1 × 10⁵ |
| 305 | II | 4.7 | 1:450 | 54 | 85 | 3.2 × 10¹⁰ |
| 101 | II | 0.32 | 1:400 | 134 | 4.1 | 3.0 × 10³ |
| 306 | III | 8.9 | 1:70 | 61 | 18.2 | 1.8 × 10⁹ |
| 307 | III | 1.5 | 1:90 | 75 | 2.9 | 1.8 × 10² |
| 102 | III | 0.26 | 1:30 | 27 | 0.2 | 7.0 × 10⁶ |
| 308 | IV | 30 | 1:30 | 19 | 29.7 | 6.5 × 10⁹ |
| 309 | IV | 0.47 | 1:20 | 186 | 0.4 | 5.6 × 10⁶ |
| 103 | IV | 3.7 | 1:10 | 79 | 1.4 | 1.2 × 10⁷ |
| 310 | V | 39 | 1:2 | 46 | 2.8 | 8.5 × 10⁶ |
| 201 | V | 40 | 1:2 | 60 | 1.9 | 6.3 × 10⁵ |
| 311 | V | 40 | 1:3.5 | 102 | 4.8 | 3.1 × 10⁷ |

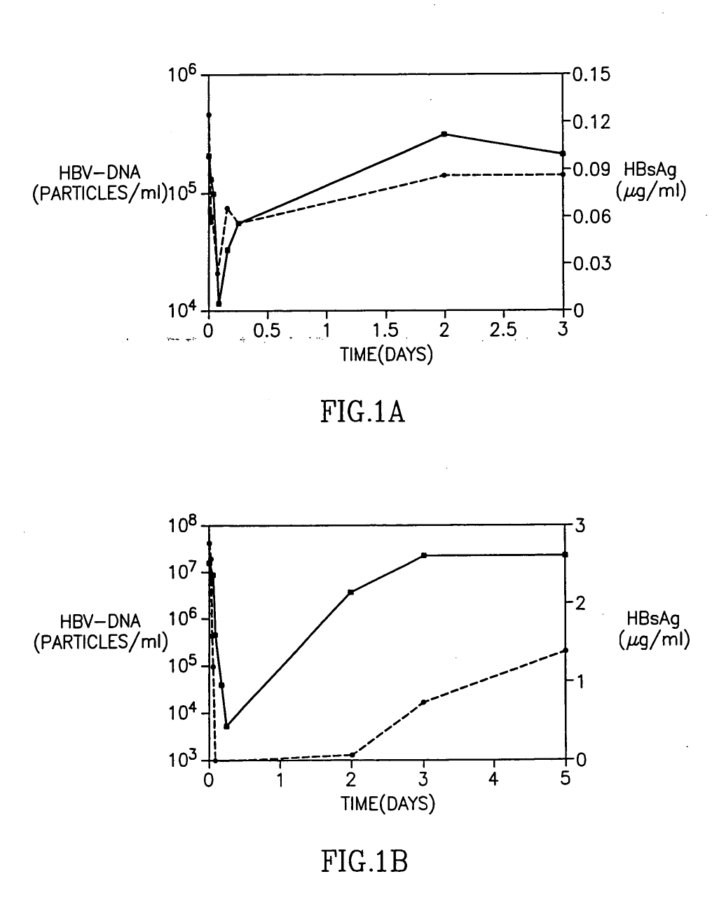

Reduction in HBsAg and HBV-DNA levels became detectable shortly after infusion initiation but was only observed in patients receiving antibodies with a high Ab:Ag ratio. In the fifth group (Ab:Ag molar ratio of 1:2) HBsAg levels decreased to undetectable levels and then started to increase 24 hr after initiation of the infusion, reaching pre-treatments levels only eight days after the infusion (FIG. 1). HBV-DNA levels also decreased after the initiation of the HBV-Ab^{XTL} infusion and reached pre-treatment levels one day later. The reduction in HBV-DNA levels was between one to three orders of magnitude. The most common adverse event reported was mild myalgia observed in six patients (40%).

### Example 2

In a subsequent, multiple-dose, dose escalation Phase IB study of patients with chronic Hepatitis B infection, 12 patients were enrolled, three patients in each of 4 sequential dose cohorts (Table 2). Each patient received 4 weekly infusions of HBV-Ab^{XTL} at doses ranging from 10 to 80 mg per infusion. The intravenous infusions were given over 2 or 4 hours.

TABLE 2
Pre-treatment clinical characterization of patients in phase 1B

| | | Dose | ALT | HBsAg | HBV-DNA |
|---|---|---|---|---|---|
| Patient | Cohort | (mg) | (U/L) | (μg/ml) | (copies/ml) |
| 303 | I | 4 × 10 | 14 | 0.02 | 2.0 × 10⁵ |
| 101 | I | 4 × 10 | 123 | 3.2 | 4.6 × 10³ |
| 304 | I | 4 × 10 | 69 | 4.4 | 4.0 × 10³ |
| 102 | II | 4 × 20 | 56 | 0.2 | 2.2 × 10⁷ |
| 302 | II | 4 × 20 | 49 | 2.7 | 4.0 × 10⁶ |
| 308 | II | 4 × 20 | 94 | 9.4 | 7.0 × 10⁸ |
| 202 | III | 4 × 40 | 19 | 41.4 | 4.0 × 108⁹ |
| 105 | III | 4 × 40 | 47 | 1.7 | 6.0 × 10³ |
| 203 | III | 4 × 40 | 38 | 1.5 | 5.0 × 10⁶ |
| 301 | IV | 4 × 80 | 137 | 4.6 | 3.0 × 10⁶ |
| 311 | IV | 4 × 80 | 120 | 5.2 | 3.0 × 10⁵ |
| 106 | IV | 4 × 80 | 87 | 0.93 | 2.0 × 10⁷ |

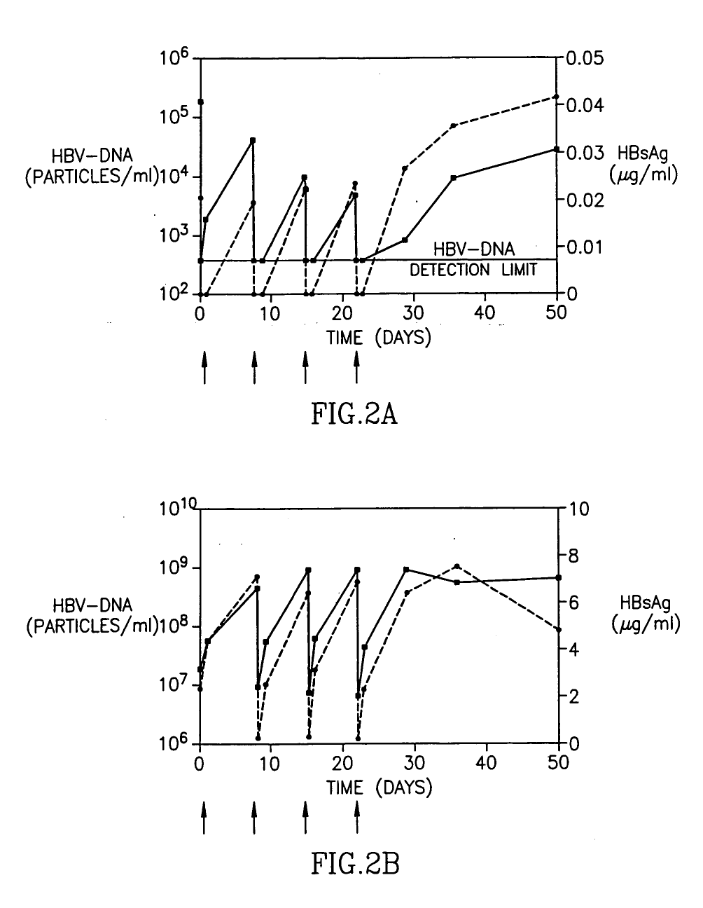

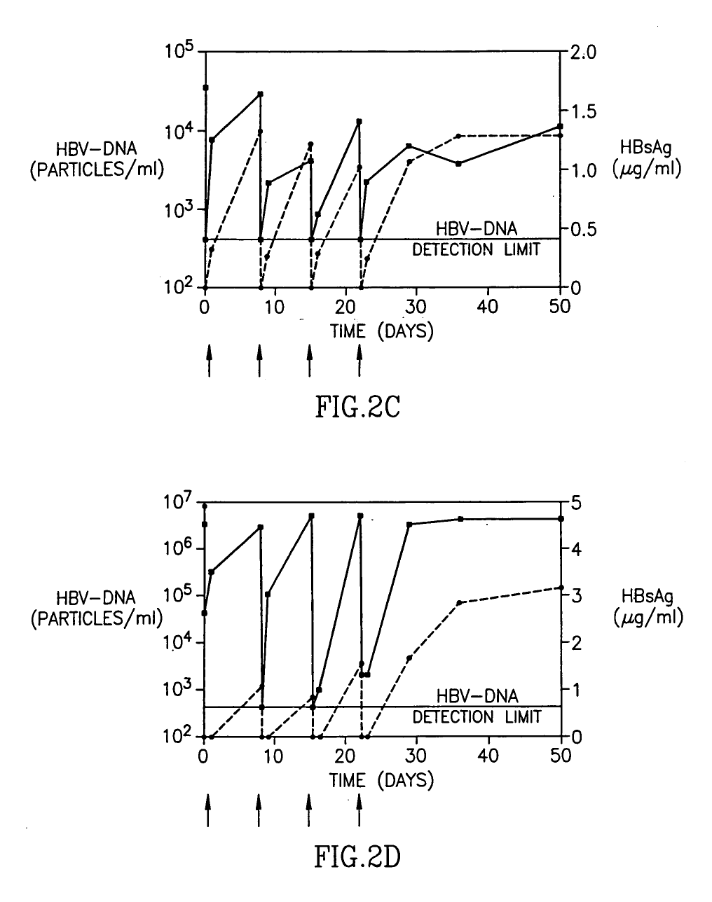

Patients from the first cohort had received 4 weekly infusions of 10 mg each. In two out of the three patients, HBsAg levels decreased to undetectable levels immediately after administration and returned back almost to the original levels prior to the next infusion. A similar pattern was observed following each administration resulting in a trend of progressive decrease in HBsAg levels during repeated administration. At 24 hours following injection, HBsAg levels were still undetectable in one patient but started to increase in the other 2 patients. Similarly, upon infusion HBV-DNA levels decreased by 3 logs and a progressive decline was observed with every administration. These levels remained undetectable for 24 hours after every infusion (FIG. 2).

The second cohort of patients received four weekly infusions of 20 mg of HBV-AB^{XTL} each (FIG. 2B). A similar pattern of reduction of HBsAg levels to undetectable limit was also observed in these three patients. HBV-DNA levels have also dropped by one to four logs. The third cohort received four weekly infusions of 40 mg of HBV-AB^{XTL} and the forth cohort received four weekly infusions of 80 mg of HBV-AB^{XTL}, each. These administrations showed similar effects on HBsAg and HBV-DNA dynamics (FIG. 2 C, D). In all cases HBV-DNA decreased significantly, and HBsAg levels were reduced to undetectable levels immediately following infusion.

The antibody was well tolerated: there were no serious adverse events and myalgia was reported in only one patient (8%). The most common adverse events were hematuria and mild chest pain, each reported in 3 out of 12 patients (25%). There was no evidence for immune complex disease.

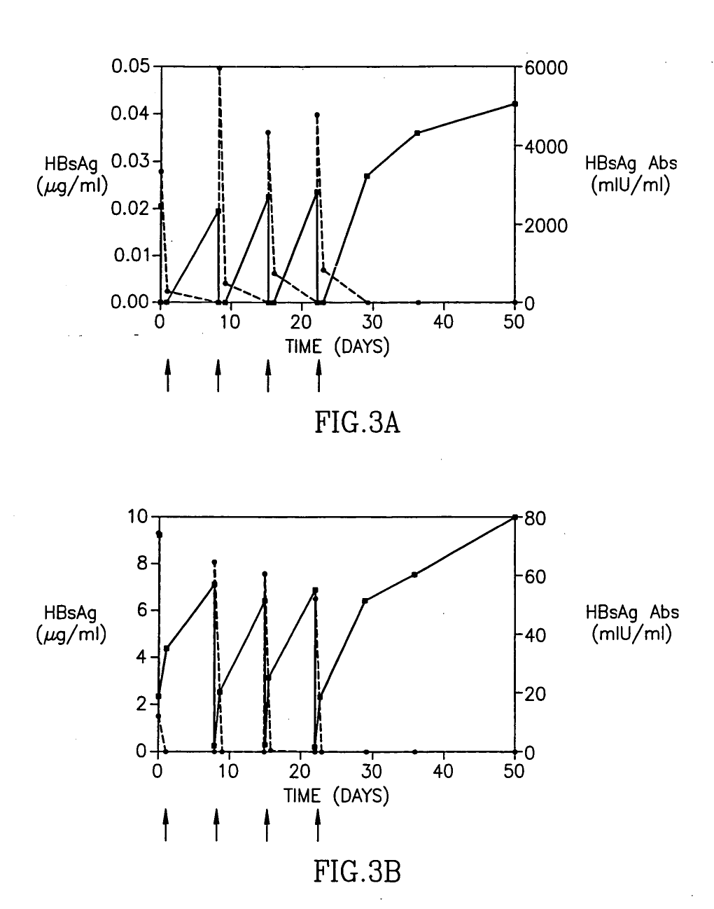

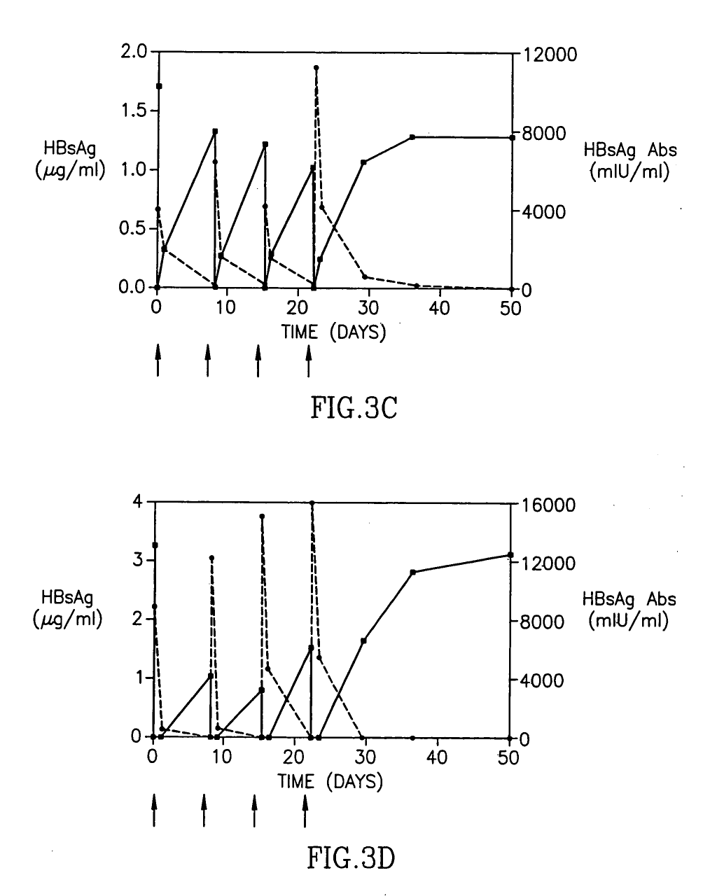

We have followed the levels of HBV-Ab^{XTL} after four weekly infusions in patients from phase 1B. The kinetics of increase and decrease of anti-HB (hepatitis B) antibody levels have opposite patterns as compared to that HBsAg levels. In all patients, after each infusion anti-HB antibody levels increased and reached a peak, then returned to pretreatment levels prior to the next administration (FIG. 3). In patients who received repeated doses of 40 mg and of 80 mg the decrease in anti-HB antibody levels was slightly slower.

### Example 3

In the following study HBV-Ab^{XTL} is given in combination with lamivudine. Lamivudine is given in a dose of 100 mg/day (The recommended dose of lamivudine for treatment of chronic hepatitis B virus infection) HBV-Ab^{XTL} is given intravenously either as a 10 mg or 40 mg dose.

The preparation of these specific doses is shown in Table 3.

TABLE 3
Amount of HBV-Ab 17.1.41 and HBV-Ab 19.79.5 in HBV-Ab^{XTL}

| | | HBV-Ab 17.1.41 | | | HBV-Ab 19.79.5 | | |
|---|---|---|---|---|---|---|---|
| Total mAb | | (2 mg/mL) | | | (1.25 mg/mL) | | |
| (mg) | (IU) | mL | mg | IU | mL | mg | IU |
| 10 | 9,310 | 3.8 | 7.6 | 4,560 | 1.9 | 2.38 | 4,750 |
| 40 | 37,240 | 15.2 | 30.4 | 18,240 | 7.6 | 9.50 | 19,000 |

Patients are treated according to the following dosing regimen:
A. HBV-Ab^{XTL} 10 mg weekly for 4 weeks followed by 10 mg every four weeks for 48 weeks plus lamivudine 100 mg once daily for 64 weeks.
B. HBV-Ab^{XTL} 40 mg weekly for 4 weeks followed by 10 mg every four weeks for 48 weeks plus lamivudine 100 mg once daily for 64 weeks.
C. HBV-Ab^{XTL} 40 mg weekly for 4 weeks followed by 40 mg every four weeks for 48 weeks plus lamivudine 100 mg once daily for 64 weeks.
D. HBV-Ab^{XTL} 40 mg three times weekly for 2 weeks, followed by 40 mg once a week for two weeks followed by 10 mg every four weeks for 48 weeks plus lamivudine 100 mg once daily for 64 weeks.
E. HBV-Ab^{XTL} 40 mg three times weekly for 2 weeks, followed by 40 mg once a week for two weeks followed by 40 mg every four weeks for 48 weeks plus lamivudine 100 mg once daily for 64 weeks.

1. A pharmaceutical composition comprising a mixture of the human monoclonal antibody 19.79.5 as well as fragments thereof retaining the antigen binding characteristics of the antibody and the human monoclonal antibody 17.1.41 as well as fragments thereof retaining the antigen binding characteristics of the antibody together with a pharmaceutically acceptable carrier.

2. A pharmaceutical composition according to claim 1 wherein the concentration of the antibodies ranges between 0.26 to 80 mg.

3. A pharmaceutical composition according to claim 1 wherein the concentration of the antibodies is 10 mg.

4. A pharmaceutical composition according to claim 1 wherein the concentration of the antibodies is 40 mg.

5. A pharmaceutical composition according to claim 1 wherein the concentration ratio in milligrams between the human monoclonal antibody 19.79.5 and the human monoclonal antibody 17.1.41 is about 1:3.

6. A pharmaceutical composition according to claim 3 comprising 2.38 mg of the human monoclonal antibody 19.79.5 and 7.6 mg of the human monoclonal antibody 17.1.41.

7. A pharmaceutical composition according to claim 4 comprising 9.5 mg of the human monoclonal antibody 19.79.5 and 30.5 mg of the human monoclonal antibody 17.1.41.

8. A pharmaceutical composition according to claim 1 in a pharmaceutical formulation effective for the treatment of hepatitis B (HBV) infection.

9. A pharmaceutical composition according to claim 1 in a pharmaceutical formulation effective for inhibiting hepatitis B infection.

10. The pharmaceutical composition according to claim 1 further comprising an anti-viral agent.

11. The pharmaceutical composition according to claim 10 wherein the anti-viral agent is selected from the group consisting of interferons, anti hepatitis B monoclonal antibodies, anti hepatitis B polyclonal antibodies, nucleoside analogues, inhibitors of DNA polymerase and therapeutic vaccines.

12. The pharmaceutical composition according to claim 10 wherein the anti-viral agent is lamivudine.

13. A method for the treatment of HBV infections comprising administering to an individual in need thereof the pharmaceutical composition according to claim 1.

14. A method for treating further HBV infections comprising administering to an individual the pharmaceutical composition according to claim 1 to prevent further infection of the treated individual with HBV.

15. A method for the treatment of HBV infections comprising administering to an individual in need thereof the pharmaceutical composition according to claim 1 in combination with an anti-viral agent.

16. A method for the treatment of HBV infections according to claim 15 wherein the anti-viral agent is selected from the group consisting of interferons, anti hepatitis B monoclonal antibodies, anti hepatitis B polyclonal antibodies, nucleoside analogues, inhibitors of DNA polymerase and therapeutic vaccines.

17. A method for the treatment of HBV infections according to claim 16 wherein the anti-viral agent is lamivudine.

18. A method for the treatment of HBV infections according to claim 15 wherein the pharmaceutical composition is given either once or three times weekly for 4 weeks and then given once every four weeks for 48 weeks in combination with a therapeutically effective amount of an anti viral agent.

19. A method for the treatment of HBV infections according to claim 18 wherein the anti viral agent is lamivudine.

20. A method for the treatment of HBV infections according to claim 19 wherein lamivudine is given once daily at a 100 mg dose.

21. A method for the treatment or prevention of HBV infections according to claim 13 wherein the pharmaceutical composition is given as a subcutaneous injection.

22. A method for the treatment or prevention of HBV infections according to claim 13 wherein the pharmaceutical composition is given as an intramuscular injection.

23. A method for the treatment or prevention of HBV infections according to claim 13 wherein the pharmaceutical composition is given as an intravenous injection.

24. A pharmaceutical composition according to claim 1 further comprising a pharmaceutical formulation effective for treating a liver transplant recipient to reduce HBV infection of a transplanted liver.

25. A pharmaceutical composition according to claims 1, further comprising a pharmaceutical formulation effective for treating an individual born to an HBV infected mother.

26. A pharmaceutical composition according to claims 1, further comprising a pharmaceutical formulation effective for treating a healthcare worker exposed to HBV.

27. A method for reducing HBV infection of a transplanted liver comprising administering to an individual in need thereof the pharmaceutical composition according to claim 24.

28. A method for treating an individual born to an HBV infected mother, comprising administering to said individual the pharmaceutical composition according to claim 25.

29. A method for treating a healthcare worker exposed to HBV, comprising administering the pharmaceutical composition according to claim 26 to said healthcare worker

30. A pharmaceutical composition comprising a first monoclonal antibody or fragment thereof comprising an amino acid sequence selected from the group consisting of SEQ ID NO: 1 and SEQ ID NO: 2 and a second monoclonal antibody or fragment thereof comprising an amino acid sequence selected from the group consisting of SEQ ID NO: 3 and SEQ ID NO: 4 and a pharmaceutically acceptable carrier.

31. The pharmaceutical composition of claim 30, wherein said first antibody comprises a variable chain sequence selected from the group consisting of the light chain variable sequence of SEQ ID NO: 1 and the heavy chain variable sequence of SEQ ID NO: 2 and wherein said second antibody comprises a variable chain sequences selected from the group consisting of the light chain variable sequence of SEQ ID NO: 3 and the heavy chain variable sequence of SEQ ID NO: 4.

32. The pharmaceutical composition of claim 31, wherein said first antibody comprises the light chain variable sequence of SEQ ID NO: 1 and the heavy chain variable sequence of SEQ ID NO: 2 and wherein said second antibody comprises the light chain variable sequence of SEQ ID NO: 3 and the heavy chain variable sequence of SEQ ID NO: 4


SEQUENCE LISTING (4 sequences)

SEQ ID NO: 1 - protein, 112 aa, Homo sapiens
DIVMTQSPLSLSVTPGEPASISCRSSQSLLHRSGNNYLDWYLQKPGHSPQLLIYVGSNRASGVPDRFSGSGSGTEYTLKISRVEAEDVGVYYCMQALQTPRTFGQGTKLEIK

SEQ ID NO: 2 - protein, 129 aa, Homo sapiens
QVQLVESGGGVVRPGRSLRLSCAASGFAFSDYSINWVRQAPGKGLEWVAIISYDGRITYYRDSVKGRFTISRDDSKNTLYLQMNSLRTEDTAVYYCARQYYDFWSGSSVGRNYDGMDVWGLGTTVTVSS

SEQ ID NO: 3 - protein, 109 aa, Homo sapiens
SYVLTQPPSVSVAPGKTARISCGGNNIGTKNVHWYQQKPGQAPVLVVYADSDRPSGIPERFSGSNSGNTATLTISRVEVGDEADYYCQVWDSVSYHVVFGGGTTLTVLG

SEQ ID NO: 4 - protein, 121 aa, Homo sapiens
QVQLVESGGGVVQPGGSLRLSCAPSGFVFRSYGMHWVRQTPGKGLEWVSLIWHDGSNRFYADSVKGRFTISRDNSKNTLYLQMNSLRAEDTAMYFCARERLIAAPAAFDLWGQGTLVTVSS